(19) United States
(12) Patent Application Publication
(10) Pub. No.: US 2009/0035398 A1
(43) Pub. Date: Feb. 5, 2009

(54) TOPICAL FORMULATIONS

(76) Inventor: Raymond Williams, Los Angeles, CA (US)

Correspondence Address: LARSON NEWMAN ABEL & POLANSKY, LLP, 5914 WEST COURTYARD DRIVE, SUITE 200, AUSTIN, TX, 78730, US

(21) Appl. No.: 12/059,103

(22) Filed: Mar. 31, 2008

Related U.S. Application Data
(60) Provisional application No. 60/908,758, filed on Mar. 29, 2007.

(51) Int. Cl. A61K 8/97; A61Q 19/00

(52) U.S. Cl. 424/735

(57) ABSTRACT

A topical formulation includes at least about 45% by weight shea butter, hemp oil in an amount not greater than 40% by weight, almond oil in an amount not greater than about 15% by weight, and jojoba oil in an amount not greater than about 15% by weight.

### CROSS-REFERENCE TO RELATED APPLICATION

The present application claims priority from U.S. Provisional Patent Application No. 60/908,758, filed Mar. 31, 2007, entitled “TOPICAL FORMULATIONS,” naming inventor Raymond Williams, which application is incorporated by reference herein in its entirety.

### FIELD OF THE DISCLOSURE

This disclosure, in general, relates to topical formulations and methods for making same.

### BACKGROUND

People generally desire a healthy look and feel to their skin. Generally, soft skin that is supple to the touch is preferred. Further, blemish free skin that has a particular glow that is not too shiny but not dull provides an appearance of health and vitality.

However, many conditions brought upon by modern living effect the look and feel of skin. For example, diseases such as diabetes may lead to chronic dryness of skin. Further, the side effects of many medications may cause dryness, scaliness, or blemishes. Also, the side effects of treatments, such as radiation or chemotherapy, may lead to undesirable skin conditions. Furthermore, the stress of modern lifestyle in conjunction with poor diet and diuretics, such as caffeine, may also produce dryness and dullness within skin.

On the other hand, diets high in fats, especially animal fats and processed trans fats, may encourage the over production of oils within skin, resulting in a shinny appearance and an oily feel to the skin. Such an oily appearance and feel is also considered undesirable. Oftentimes, to counteract an oily appearance, people turn to soaps and detergents that are often either ineffective or cause the opposite problem, dry and scaly skin.

Further, chronic dry skin may build up causing calloused regions, such as on the hands, on the elbows, on the feet, and on other locations of the body. In some cases, dry skin clogs pores, causing pimples and other blemishes.

As such, many traditional treatments for skin do not produce the desired moisture balance indicative of healthy skin.

### DESCRIPTION OF THE EMBODIMENTS

In a particular embodiment, a topical formulation includes shea butter, hemp oil, almond oil, and jojoba oil. The topical formulation also may include a fragrant component, such as an essential oil. Such a topical formulation has a desirable texture and spreading profile. Further, when used as a topical ointment, such a topical formulation results in skin that strikes a desirable balance between oily and dry. In an exemplary embodiment, the topical formulation is produced by blending shea butter, hemp oil, almond oil, and jojoba oil at temperatures between about 15° C. and about 3° C.

In an additional embodiment, an exfoliating formulation includes crystalline salt derived from an ancient dried sea source and flora derived oil including at least one of hemp oil, sweet almond oil, or olive oil. The exfoliating formulation also may include a fragrant component, such as an essential oil. Such an exfoliating formulation is useful in exfoliating dry skin and treating callused regions. The exfoliating formulation may be blended at temperatures between about 15° C. and about 30° C.

In a particular embodiment, a topical formulation includes shea butter. Shea butters are generally available in various grades, including raw or unrefined, refined, or bleached. Raw or unrefined shea butter is derived from a Butyrospermum parkii nut through crushing and pressing. To refine the unrefined shea butter, the shea butter is generally heated causing the shea butter to melt. The refining process permits straining and decanting of the shea butter to reduce the presence of dirt and other particulate. The shea butter may be chemically processed or water washed, resulting in a bleached shea butter that is generally clear or white. Once the shea butter is treated through heating, it is generally recrystallized, giving the shea butter a grainy texture or a waxy texture. Typically, the refining process and the bleaching process alter the trace compounds found in the shea butter, such as the anti-oxidants, natural colorants, and other compounds that provide raw or unrefined shea butter with a unique texture and color.

In a particular example, the shea butter used in the topical formulation is a raw or unrefined shea butter, such as shea butter that is not heat treated after extraction from the nut. In particular, the shea butter may be a cold pressed unrefined shea butter.

In addition, the topical formulation includes a set of flora-derived oils. In particular, the topical formulation includes hemp oil, almond oil, and jojoba oil. In particular embodiments, olive oil may be substituted for hemp oil. In an example, the hemp oil is a cold pressed hemp oil. In another example, the almond oil is a sweet almond oil, such as a cold pressed sweet almond oil. In a further example, the jojoba oil is a cold dark pressed jojoba oil. Generally, the topical formulation is alcohol free.

In an exemplary embodiment, a topical formulation comprises at least about 45% by weight shea butter. For example, the topical formulation may include shea butter in an amount of greater than about 60% by weight, such as an amount of at least about 80% by weight, or even an amount of at least about 90% by weight.

In addition, the topical formulation includes hemp oil in an amount not greater than about 40% by weight. For example, hemp oil may be included in an amount of about 5% to about 35% by weight, such as an amount of about 20% to about 35% by weight. In a particular example, the hemp oil is included in an amount of about 30% to about 35% by weight. In another example, the hemp oil is included in an amount of about 20% to about 25% by weight.

In an exemplary embodiment, the almond oil is included in an amount not greater than about 15% by weight. For example, the almond oil may be included in an amount of about 5% to about 15% by weight, such as an amount of about 9% to about 12% by weight. Similarly, the jojoba oil may be included in an amount not greater than about 15% by weight. For example, the jojoba oil may included in an amount of about 5% to about 15% by weight, such as an amount of about 9% to about 12% by weight.

The topical formulation may further include a fragrant component, such an essential oil or an extract. An exemplary fragrant component include an extract or essential oil derived from lavender, patchouli, rosemary, chamomile, rosebud, orange blossom, St. John's wart, eucalyptus oil, jasmine, peppermint, thyme, myrtle, myrrh, rose, teatree oil, pine, fruit, vanilla, bergamot essential oil, or any combination thereof. In a particular embodiment, the fragrant component is an essential oil, such as a certified organic essential oil.

In a particular example, the topical formulation includes a fragrant component, such as an essential oil, in an amount of not greater than about 5% by weight. For example, the essential oil may be included in an amount not greater than 3% by weight, such as an amount not greater than about 1% by weight, not greater than about 0.5% by weight, or even not greater than about 0.1% by weight.

In a particular embodiment, the topical formulation consists essentially of at least about 45% by weight shea butter, hemp oil or olive oil in an amount not greater than about 40% by weight, almond oil in an amount not greater than about 15% by weight, jojoba oil in an amount not greater than about 15% by weight, and an essential oil in an amount not greater than about 5% by weight. While a range of weight percent may be specified, it is recognized that the sum of all weight percents add to 100%. A change in the weight percent of any one component results in a corresponding change to other weight percents to maintain a total weight percent of 100%.

In an exemplary embodiment, the topical formulation may be formed by blending the ingredients at low temperatures. For example, a topical formulation may be formed by blending shea butter, hemp oil, almond oil, and jojoba oil at a temperature between about 15° C. and about 30° C. to form a blend. For example, the topical formulation may be formed by blending at a temperature between about 20° C. and 30° C., such as a temperature of about 22° C. to about 28° C. Blending may be performed for at least 30 minutes, such as at least about 1 hour. In a particular example, the components may be blended for about 1 hour to about 3 hours, such as about 1 hour to about 2 hours.

The method further may include blending a fragrant component, such as an essential oil, to form the blend. In a particular example, the method includes blending at least about 45% by weight shea butter, not greater than about 15% by weight almond oil, not greater than about 15% by weight jojoba oil, and not greater than about 35% by weight hemp oil. The method also may include blending not greater than about 5% by weight of the fragrant component, such as an extract or essential oil. In particular, the blending may include blending unrefined shea butter.

In a particular example, the topical formulation may have a desirable consistency. For example, the topical formulation may have a Consistency of about 10 grams to about 100 gram as determined by the testing method described in Example 1 below. For example, the topical formulation may have a Consistency of about 15 grams to about 80 grams, such as about 20 grams to about 80 grams.

Further, the topical formulation may have a desirable density. For example, the topical formulation may have a specific gravity not greater than about 0.93, such as not greater than about 0.9, not greater than about 0.8, or even not greater than about 0.75.

Particular embodiments of the above topical formulations provide advantageous technical features. In a particular example, cold processing of shea butter topical formulations that include unrefined shea butter provide an aesthetic color and a pleasing texture not found in those topical formulations that include refined shea butters and formed at high temperatures. Often, traditional shea butter formulations are combined at temperatures greater than 30° C. Such processing changes the texture and appearance of the final product and may lead to a reduced shelf life. In contrast, cold processing of topical formulations that include unrefined shea butter unexpectedly provides a product with an aesthetic visual appeal and a smooth and creamy texture compared to traditionally formed products. Further, the embodiments exhibit an extended shelf life.

Unexpectedly, such a raw or unrefined shea butter imparts in the topical formulation a desirable color that is aesthetically pleasing to the consumer. Further, the raw or unrefined shea butter may impart a desirable texture to the topical ointment that is smooth and creamy to the touch without being oily. Moreover, the raw or unrefined shea butter also provides the topical formulation with a longer shelf life. Such a longer shelf life is generally unexpected, because refining and bleaching is typically used to improve the shelf life of animal or plant byproducts.

In another exemplary embodiment, an exfoliating formulation may include a crystalline salt derived from an ancient dried sea source and flora derived oil. For example, the flora-derived oil may include at least one of hemp oil, sweet almond oil, or olive oil. In a particular example, the flora-derived oil is a sweet almond oil that is cold pressed.

The crystalline salt is selected from those salts that are derived from ancient dried seas or oceans. In particular, the salt source is an ancient salt-water body that dried at least one thousand years ago. In an example, the crystalline salt is a Himalayan pink salt. One commercial vendor of Himalayan pink salt is tropicaltraditions.com. In another example, the crystalline salt is a Redmond Real Salt. One commercial vendor of Redmond Real Salt is pleasanthillgrain.com.

In an example, the exfoliating formulation may include crystalline salt in an amount of at least about 50% by weight, such as an amount of at least about 70% by weight. The flora-derived oil may be included in an amount not greater than about 50% by weight, such as an amount not greater than about 30% by weight, not greater than about 15% by weight, or not greater than about 10% by weight.

The exfoliating formulation may include a fragrant component. In an example, the fragrant component is an essential oil or extract such as those fragrant components disclosed above. Alternatively, the fragrant component may be an extract. In an example, the exfoliating formulation includes the fragrant component in an amount not greater than about 5% by weight, such as an amount not greater than 1% by weight, not greater than an amount 0.5% by weight, or not greater than 0.1% by weight.

The exfoliating formulation may be formed through blending the crystalline salt and the flora-derived oil at low temperatures. For example, the salt and oils may be blended at temperatures between about 15° C. and about 30° C., such as about 20° C. and about 30° C.

Particular embodiments of the exfoliating formulation provide unexpected technical advantages. For example, users of the exfoliating formulation report a decrease in blemishes, such as pimples.

### Example 1

Comparative testing of the shea butter topical formulation and commercially available shea butter products is performed. Consistency is tested using the testing method below. Such a testing method is developed to provide a result that more closely relates to the experience of the consumer when comparing product samples in a store.

An open container including a product is placed on a Terraillon® CB5 scale having a maximum capacity of 5 kg. The scale is tarred. A round wooden toothpick available from Diamond® is inserted 2 cm into the product using a single finger placed at the top of the toothpick. A maximum weight in grams is measured during insertion. Table 1 illustrates the results of testing a shea butter topical formulation and commercially available shea butter products.

TABLE 1
Consistency of Products

| Sample | Description | Consistency (g) |
|---|---|---|
| Example Topical | Topical formulation | 50-60 |
| Formulation | including shea, hemp, | |
| | almond, and jojoba oils | |
| True Blue ® Spa too | 100% shea butter balm | 110-120 |
| shea ™ | available from Bath & | |
| | Body Works | |
| True Blue ® Spa | Soft body butter with | 5-10 |
| mega butter ™ | four natural butters | |

The example topical formulation exhibits a desirable intermediate consistency. The too Shea™ product exhibits a consistency that is high and the mega Butter™ product exhibits a consistency that is low.

Specific gravity is also tested. The mega Butter™ product exhibits a specific gravity of approximately 0.98-0.99. The too Shea™ product exhibits a specific gravity of approximately 0.91-0.93. The example topical formulation exhibits a consistency that is less than 0.9, and is approximately 0.75 to 0.8.

### Example 2

A diabetic individual suffers from chronic dry skin. The individual traditionally uses Eucerin® DRY SKIN THERAPY Original Moisturizing Crème to treat the condition. For treatment of the skin, the man replaces the Eucerin® with the example shea butter topical formulation and notices a reduction in reappearance of the dry skin condition and a reduction in the frequency of application of skin treatment.

### Example 3

An individual with plantar hyperhidrosis experiences a side effect of itching cracked skin between toes. The individual applied the example shea butter topical formulation once a day for three days to the feet before bed. The individual reports a reduction in the severity of the side effect.

The above-disclosed subject matter is to be considered illustrative, and not restrictive, and the appended claims are intended to cover all such modifications, enhancements, and other embodiments, which fall within the true scope of the present invention. Thus, to the maximum extent allowed by law, the scope of the present invention is to be determined by the broadest permissible interpretation of the following claims and their equivalents, and shall not be restricted or limited by the foregoing detailed description.

1. A topical formulation comprising:
at least about 45% by weight shea butter;
hemp oil in an amount not greater than 40% by weight;
almond oil in an amount not greater than about 15% by weight; and
jojoba oil in an amount not greater than about 15% by weight.

2. The topical formulation of claim 1, further comprising an essential oil in an amount not greater than about 5% by weight.

3. (canceled)

4. (canceled)

5. (canceled)

6. The topical formulation of claim 1, wherein the shea butter is unrefined shea butter.

7. The topical formulation of claim 1, wherein the hemp oil is cold pressed hemp oil.

8. The topical formulation of claim 1, wherein the almond oil is sweet almond oil.

9. The topical formulation of claim 8, wherein the sweet almond oil is cold pressed sweet almond oil.

10. The topical formulation of claim 1, wherein the jojoba oil is cold dark pressed jojoba oil.

11. The topical formulation of claim 1, wherein the shea butter is included in an amount of at least about 60% by weight.

12. The topical formulation of claim 11, wherein the shea butter is included in an amount of at least about 80% by weight.

13. The topical formulation of claim 1, wherein the hemp oil is included in an amount of about 5% to about 35% by weight.

14. The topical formulation of claim 13, wherein the hemp oil is included in an amount of about 20% to about 35% by weight.

15. The topical formulation of claim 14, wherein the hemp oil is included in an amount of about 30% to about 35% by weight.

16. The topical formulation of claim 14, wherein the hemp oil is included in an amount of about 20% to about 25% by weight.

17. The topical formulation of claim 1, wherein the almond oil is included in an amount of about 5% to about 15% by weight.

18. (canceled)

19. The topical formulation of claim 1, wherein the jojoba oil is included in an amount of about 5% to about 15% by weight.

20. (canceled)

21. The topical formulation of claim 1, wherein the topical formulation is free of alcohol.

22. A topical formulation consisting essentially of:
at least about 45% by weight shea butter;
hemp oil or olive oil in an amount not greater than 40% by weight;
almond oil in an amount not greater than about 15% by weight;
jojoba oil in an amount not greater than about 15% by weight; and
an essential oil in an amount not greater than about 5% by weight.

23. A method of forming a topical formulation, the method comprising:
blending shea butter, hemp oil, almond oil, and jojoba oil at a temperature between about 15° C. and about 30° C. to form a blend.

24. The method of claim 23, further comprising blending an essential oil to form the blend.

25. (canceled)

26. (canceled)

27. (canceled)

28. (canceled)

29. The method of claim 23, wherein blending includes blending unrefined shea butter.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)
